(19) United States
(12) Patent Application Publication
(10) Pub. No.: US 2025/0044276 A1
(43) Pub. Date: Feb. 6, 2025

(54) Chemoresponsive Dyes and Chemiresisive Sensors for Rapid Assay of Scent

(71) Applicant: Trustees of Tufts College, Medford, MA (US)

(72) Inventor: Sameer Sonkusale, Lincoln, MA (US)

(21) Appl. No.: 18/717,087

(22) PCT Filed: Dec. 7, 2022
(86) PCT No.: PCT/US2022/052088

§ 371 Date: Jun. 6, 2024

Related U.S. Application Data
(60) Provisional application No. 63/286,771, filed on Dec. 7, 2021.

(51) Int. Cl. G01N 33/497; G01N 21/78; G01N 27/12

(52) U.S. Cl. CPC G01N 33/4975; G01N 21/783; G01N 27/12

(57) ABSTRACT

A sensor array that is configured to distinguish between volatilomes, each of which comprises a plurality of volatile organic compounds, includes a fibrous substrate with sensors disposed thereon. Some of the sensors change color in response to exposure to particular volatile organic compounds and others change resistance in response to exposure to particular volatile organic compounds. These volatile organic compounds are collected by a patch that is placed on the skin of a subject and then heated in the presence of the sensors.

### RELATED APPLICATIONS

This application claims the benefit of the Dec. 7, 2021 priority date of U.S. Provisional Application 63/286,771, the contents of which are incorporated herein by reference.

### FIELD OF INVENTION

This invention pertains to detecting molecules, and in particular, to detecting molecules of volatile organic compounds.

### BACKGROUND

In the course of daily life, a human emits a characteristic scent. This scent is analogous to a fingerprint for a particular human. This scent is sufficiently distinctive to permit bloodhounds to track down a particular human fugitive through a forest.

The human scent arises from various molecules of volatile organic compounds. In some cases, these molecules are emitted intermittently, for example during a bout of flatulence.

However, in other cases, these molecules are emitted more or less continuously. Examples of the latter include molecules emitted from the skin or through the saliva. Both of these sources contribute to the human's scent. The combination of the scent's constituents is often called the human volatilome.

The human volatilome has applications well beyond tracking escaped fugitives. In fact, it is also indicative of the condition of the human's medical condition. An obvious example is the distinctive ammonia-like scent that arises when a human's kidneys fail to excrete urea quickly enough to avoid accumulation. Another example is the characteristic scent that arises when certain metabolic processes change during aging.

A human's scent is a combination of different volatile organic compounds in different amounts. It is therefore useful to be able to identify those compounds and their respective concentrations. Doing so provides a non-invasive basis for analysis of the human's condition.

### SUMMARY

In one aspect, the invention features a patch that is configured to collect volatile organic compounds from a subject and a sensor array that is in gaseous communication with that patch. The sensor array is configured to distinguish between volatilomes, each of which comprises a plurality of volatile organic compounds. Such a sensor array includes a fibrous substrate having first and second sets of sensors disposed thereon. Sensors in the first set of sensors, referred to herein as “first sensors” or “colorimetric sensors” change color in response to exposure to particular volatile organic compounds. Meanwhile, sensors in the second set of sensors, referred to herein as “second sensors” or “chemiresistive sensors,” change resistance in response to exposure to particular volatile organic compounds. As a result, after having absorbed volatile organic compounds, the patch releases those compounds, thereby exposing the sensor array to those compounds.

Embodiments include those in which the patch comprises a microporous polymer that serves as a porous thin-film for extraction of volatile organic compounds, those in which it comprises a hydrophobic polymer, those in which it comprises a hydrophilic polymer, and those in which it comprises a material selected to capture a non-polar gas.

Still other embodiments include those in which the patch comprises PDMS and those in which it comprises polyacrylate.

Embodiments include those in which the colorimetric sensors comprise dyes that change color in response to exposure to a particular volatile organic compounds and those in which the chemiresistive sensors comprise materials that change resistance in response to exposure to particular volatile organic compounds.

Also among the embodiments are those in which the fibrous substrate comprises paper and those in which it comprises textile.

Other embodiments feature a color reference disposed on the fibrous substrate. This color reference comprises samples of colors that match particular states of the colorimetric sensors.

Still other embodiments include those in which electrodes that are disposed on the substrate are connected to the chemiresistive sensors.

Among the embodiments are those that include a heater configured to heat the patch thereby causing the patch to release the volatile organic compounds. Examples of heaters include a thermoelectric heater, a resistive heater, an induction heater, and a solar heater.

In still other embodiments, the apparatus includes an patch and a heater. The patch collects volatile organic compounds. Such collection arises, for example, by adsorption or by absorption. After having sampled a subject's volatilome, the patch is disposed between the heater and the sensor array. The patch is then heated. This releases the volatile organic compounds captured by the patch so that they can interact with the sensors in the sensor array.

Also among the embodiments are those that include a portable instrument that reads resistances of the chemiresistive sensors and views colors associated with the first sensors and those that include a smart phone having a camera that is disposed such that the colorimetric sensors are in the camera's field-of-view.

Still other embodiments include a support vector machine that receives information indicative of which of the colorimetric and chemiresistive sensors have undergone interaction in response to exposure to a sample of volatile organic compounds, the support vector machine having been trained to recognize a condition associated with the information. Examples of conditions include an infection by a virus, such as Covid-19 or variants thereof.

Another example of a condition to be identified is fatigue, for which there is currently no single point-of-care diagnostic device. The apparatus described herein relies on broad-spectrum metabolomics to identify chemical signatures of fatigue, and in particular, signatures that arise from observing profiles of volatile organic compounds.

The invention provides a portable platform for detecting volatile organic compounds emanating from skin and/or saliva and to associate these profiles of volatile organic compounds to patients with a variety of conditions, including symptomatic or asymptomatic covid-19. The platform provides a low-cost optical and electronic “nose” that is based on a macro-porous polymeric sampler that collects volatile organic compounds from skin and saliva with high extraction efficiency and that also subsequently analyzes the collected volatile organic compounds by a cross-reactive chemical sensor array fabricated on a paper substrate. Since the sense of smell is in large part the detection of volatile organic compounds, the resulting is, in effect, a “paper nose.”

As a result of its ability to easily and conveniently monitor volatile organic compounds emitted from the skin and from saliva, the platform creates a comprehensive profile of volatile organic compounds, i.e., a volatilome. This, in turn, translates into increased robustness in the detection of a particular condition across various demographics groups. Examples of conditions include infections from microorganisms, including viruses such as the covid virus, fatigue, and any other condition having a signature that is determinable by observing a profile of volatile organic compounds.

In some embodiments, a portable cost-effective instrument for such analysis comprises a hot-plate for programmed thermal desorption and a smartphone for readout from both the optical sensors and the chemiresistive sensors.

Among the embodiments are those that include a privacy-aware and robust engine for identifying volatile organic compounds in a manner similar to that carried out by a mammalian olfactory system. In some embodiments, such an engine is an artificial intelligence engine. In other embodiments, such an engine is a machine-learning engine. In either case, the engine accurately classifies the ensemble response of the sensor array to distinguish between those who have the target condition and those who do not. In some embodiments, the target condition is the existence of a COVID-19 infection. In others, the target condition is fatigue of the type having a signature defined by a profile of volatile organic compounds.

A suitable sensor array comprises a wearable macro-porous sampler for collection of volatile organic compounds from the skin and saliva with high extraction efficiency and reduced sampling time and a paper-based cross-reactive sensor array using diverse set of chemo-responsive dyes and chemiresistive nanomaterials as odor receptors.

Still other embodiments include those that comprise portable instrumentation for desorption of volatile organic compounds and colorimetric readout and those that comprise a portable instrumentation using smartphone for optical sensor readout and an electronic accessory for chemiresistive sensor readout.

Further embodiments include a support vector machine and a multilayer neural-network based classifiers to process the data from the sensor array for reliable classification with privacy and fairness guarantees.

In another aspect, the invention features a method that includes providing a machine-learning system that has been trained to use a profile of volatile organic compounds as a basis for determining the existence of a target condition, collecting volatile organic compounds from a subject onto a patch, providing a sensor array that comprises colorimetric sensors and chemo-resistive sensors disposed on fibrous substrate, exposing the sensor array to the volatile organic compounds, and determining a volatilome of the subject based on a color change in the colorimetric sensors and a resistance change in the chemiresistive sensors.

Practices include those in which one uses a smart phone to observe the color change.

Also among the practices are those that include enclosing the sensor array and the patch in an enclosed space and heating the patch within the enclosed space to promote evaporation of the volatile organic compounds.

The paper-nose complements traditional virus and antibody detection to monitor the onset, progression, and resolution of a target condition, such as COVID-19. The platform is low risk to the patient and the caregiver as it involves collection of saliva in a collection tube and/or the use of a band-aid like adsorbent patch to acquire volatile organic compounds from the skin. Since no sample preparation or experience is required, any user armed with the companion smartphone app can operate the instrument. Lack of invasiveness, easy manufacturability, use of low-cost materials and reagents, and leveraging of the smartphone for readout ensures adherence to the World Health Organization's ASSURED criteria for developing medical diagnostics that can be deployed globally.

These and other features of the invention will be apparent from the following detailed description and the accompanying figures, in which:

### BRIEF DESCRIPTION OF THE FIGURES

FIG. 1 shows a portion of a sensor;

FIG. 2 shows a sensor being placed on an absorbing pad disposed on a heater;

FIG. 3 shows the heater being used to desorb volatile organic compounds on the absorbing pad to thereby expose the sensor to those organic compounds;

FIG. 4 shows the sensor array of FIG. 1 with additional features to permit reading of data; and

FIG. 5 shows an implementation of a device for reading the sensor of FIG. 1.

### DETAILED DESCRIPTION

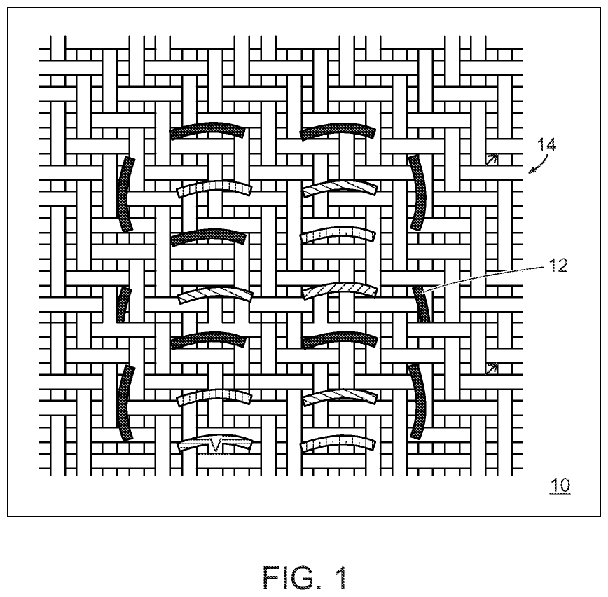

FIG. 1 shows a portion of a sensor array 10 formed from a fibrous substrate, such as paper or fabric. The sensor array 10 includes colorimetric sensors 12 and chemiresistive sensors 14. The colorimetric sensors 12 comprise dyes that change color when exposed to particular gases. The chemiresistive sensors 14 comprise materials that change resistivity in response to exposure to certain gases.

A sensor array 14 includes different kinds of both colorimetric sensors 12 and chemiresistive sensors 14, each of which is tuned to respond to a particular volatile organic compound. As a result, by observing patterns of changes in the sensors 12, 14, it is possible to infer the constituents of the gas mixture to which the sensor array 10 has been exposed.

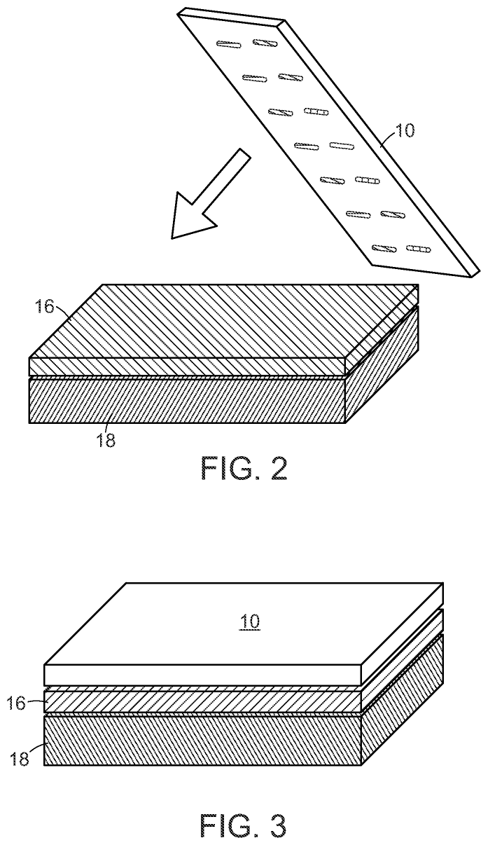

Referring now to FIG. 2, the gas mixture is sampled using an absorbent patch 16 that is placed on the human subject. A suitable patch 16 is a disposable patch that comprises a microporous PDMA sampler. Other examples include a patch configured for thin-film microextraction. A polydimethylsiloxane adsorbent is particularly useful for its high surface-to-volume ration, which enables rapid sampling compared to bulk PDMS adsorbent and an increased overall extraction efficiency.

After having been removed from the subject, the patch 16 is placed on a heater 18. The sensor array 10 is then placed on top of the absorbent patch 16. FIG. 2 shows this step in the process of completion. This results in the structure shown in FIG. 3. Although FIG. 3 shows a relatively small spacing between the patch 16 and the sensor array 10, this is not necessary. What matters is that volatile organic compounds make their way in sufficient numbers from the patch 16 to the sensor array 10.

In operation, heat from the heater 18 promotes release of molecules of volatile organic compounds that have been collected by the patch 16. These molecules then travel to the sensor array 10 and interact with the various sensors contained therein. The sensor array 10 is then read with suitable equipment.

Reading the colorimetric sensors 12 amounts to observing their respective colors.

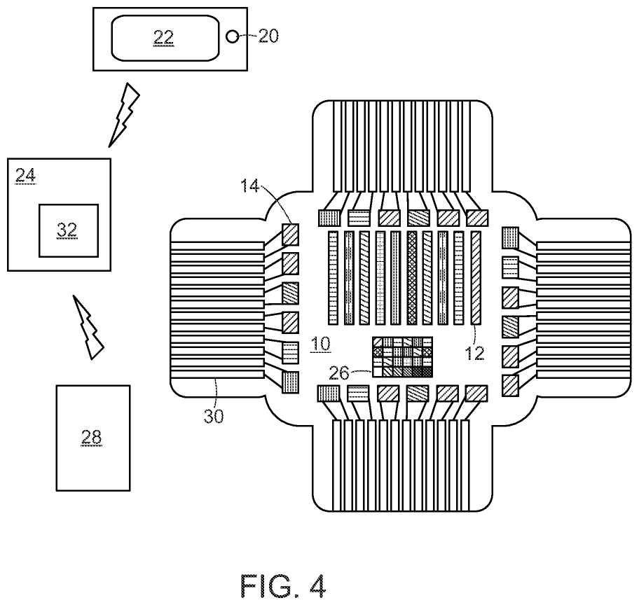

Referring now to FIG. 4, this reading operation is carried out by a camera 20 such as that available on a smartphone 22. The resulting image, once captured, is sent to a data-processing system 24 that has information indicative of which sensors 12 are used to sense which molecules and what colors correspond to which volatile organic compounds. To correct for differences between cameras 20 and to adjust for lighting, it is useful to provide a color reference 26 in the camera's field of view.

The chemiresistive sensors 14 are read by a resistance reader 28 having terminals that engage corresponding terminals 30 that lead to corresponding chemiresistive sensors 14. The resistance reader 28 then applies a known voltage across the chemiresistive sensors 14 and observes the current that results. The resulting data is likewise transmitted to the data-processing system 24.

The data-processing system 24 includes a machine-learning system for training a classifier 32. By collecting volatilome samples from various humans with known conditions, it is possible to train the classifier 32 to recognize those conditions based on their characteristic volatilome. In some embodiments, the classifier 32 has been trained to identify the signature volatilome associated with viral infections, and in particular, coronavirus infections.

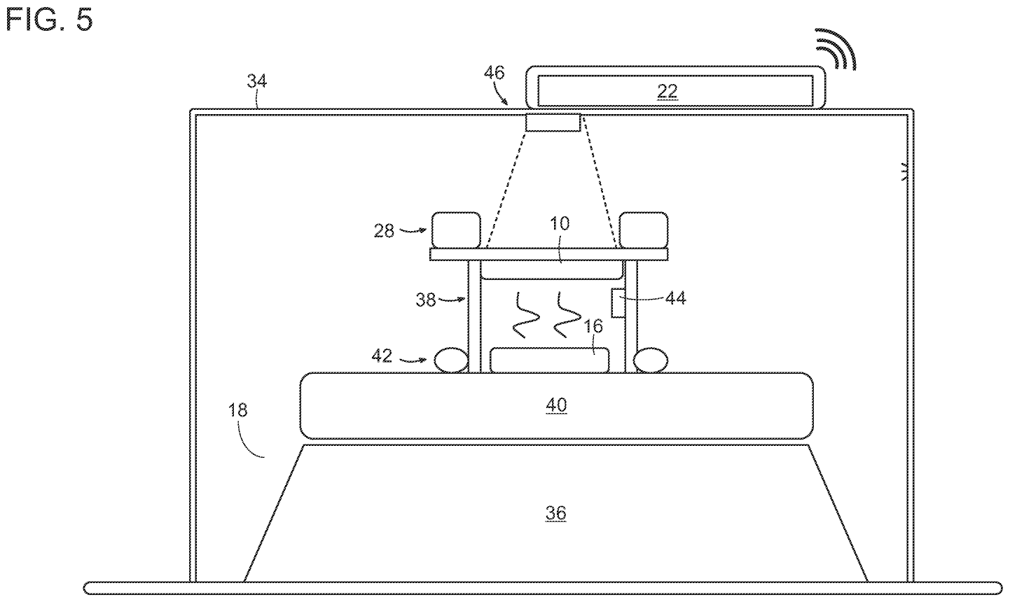

Referring now to FIG. 5, an outer chamber 34 blocks background light and promotes a stable environment that is separate from the exterior environment. Some embodiments feature a source of inert gas, such as nitrogen, to flush the interior of the outer chamber 34 before use.

The outer chamber 34 encloses the heater 18. In the illustrated embodiment, a programmable controller 36 controls the temperature of a heat source 40. Examples of a heat source include devices that convert non-thermal energy into thermal energy. Such devices include a hotplate, a thermoelectric heater, a resistive heater, an induction heater, and a solar heater. The controller 36 is particularly advantageous because it is able to control the temperature as a function of time, thus permitting the heater 18 to heat according to a temperature ramp up to some maximum temperature. A suitable temperature for many such compounds of interest is approximately 250° C.

The use of a temperature ramp enables fractional release of different gases based on their volatility and relative affinities for the sensors 12, 14, thus enabling the sensor array 10 to function in a manner analogous to an analytic column. Since different gases desorb at different rates, the result will be a color and resistance change over time. Both color and resistance are thus measured by a ratio of a differential change to a baseline value. A color change exists for each of the three primary colors and is represented by a differential intensity normalized by a baseline intensity for each primary color. Similarly, the resistance reader 28 provides a differential resistance normalized by a baseline resistance. A suitable resistance reader 28 features an onboard impedance analyzer connected via multiplexers.

An inner chamber 38 that rests on the heat source 40 encloses the patch 16, which is also on the heat source 40. A suitable material for the inner chamber 38 is glass, and in particular, a heat-resistant glass, such as PYREX. Insulation tape 42 around the base of the inner chamber 38 provides a seal that ensures efficient collection of the evaporated volatile organic compounds. The inner chamber 38 is optimized to provide a high concentration of volatile organic constituents but without saturating the sensor array 10 while also preventing excessive temperatures at the sensor array 10.

An environmental sensor 44 comprises a temperature sensor, humidity sensor, and pressure sensor. The temperature sensor provides feedback control over the controller 36 so that it can maintain a suitable temperature ramp. The pressure sensor provides a basis for inferring how much volatile gas has been released. The humidity sensor provides a basis for inferring total evaporative water loss. These latter two measurements are useful for eliminating bias across demographics during machine learning.

The resistance reader 28 for the chemiresistive sensors 14 is disposed on the inner chamber 38 so as to make contact with the sensor array 10.

The outer chamber 34 features an aperture through which the camera 20 of the smartphone 22 views the sensor array 10. A ring-light 46 illuminates the sensor array 10 to enable the camera to see the colors on the colorimetric sensors 12. In some embodiments, the ring-light 46 also emits ultraviolet radiation for UV-assisted desorption.

Additional embodiments include slots for holding emission filters, thereby permitting multi-spectral imaging using a monochrome CCD camera.

Examples of colorimetric sensors 12 include those that rely on chemo-responsive organic dyes such as solvatochromic dyes, pH indicator dyes, and porphyrins. Examples of chemo-resistive sensors 14 include those based on carbon-based nanomaterials, such as carbon nanotubes and reduced graphene oxide, organic polymers, such as PEDOT: PSS and polyalanine, and metal-oxide nanowires, such as tin oxide, tungsten oxide, vanadium oxide, an manganese oxide.

Having described the invention and a preferred embodiment thereof, what is claimed as new and secured by Letters Patent is:

1. An apparatus comprising a patch and a sensor array, wherein said patch is configured to collect volatile organic compounds from a subject, wherein said sensor array is in gaseous communication with said patch, wherein said sensor array is configured to distinguish between volatilomes, each of which comprises a plurality of volatile organic compounds, wherein said sensor array comprises a fibrous substrate, colorimetric sensors, and chemiresistive sensors, said colorimetric sensors and chemiresistive sensors being disposed on said fibrous substrate, wherein each of said colorimetric sensors undergoes a change in color in response to having been exposed to a corresponding one of said volatile organic compounds, and wherein each of said chemiresistive sensors undergoes a change in electrical resistance in response to having been exposed to a corresponding one of said volatile organic compounds.

2. The apparatus of claim 1, wherein said patch comprises a microporous polymer that serves as a porous thin-film for extraction of said volatile organic compounds.

3. The apparatus of claim 1, wherein said patch comprises PDMS.

4. The apparatus of claim 1, wherein said patch comprises a hydrophobic polymer.

5. The apparatus of claim 1, wherein said patch comprises a material selected to capture a polar gas.

6. The apparatus of claim 1, wherein said patch comprises a hydrophilic polymer.

7. The apparatus of claim 1, wherein said patch comprises a polyacrylate.

8. The apparatus of claim 1, wherein said patch comprises a material selected to capture a non-polar gas.

9. The apparatus of claim 1, wherein each of said colorimetric sensors comprises a dye that changes color in response to exposure to a particular volatile organic compound.

10. The apparatus of claim 1, wherein said chemiresistive sensors comprise materials that change resistance in response to exposure to particular volatile organic compounds.

11. The apparatus of claim 1, wherein said fibrous substrate comprises paper.

12. The apparatus of claim 1, wherein said fibrous substrate comprises textile.

13. The apparatus of claim 1, further comprising a color reference disposed on said fibrous substrate, said color reference comprising samples of colors that said colorimetric sensors change into upon exposure to corresponding ones of said corresponding volatile organic compounds.

14. The apparatus of claim 1, further comprising electrodes connected to said chemiresistive sensors.

15. (canceled)

16. The apparatus of claim 1, further comprising a heater configured to cause desorption of said volatile organic compounds from said patch after said patch has collected said volatile organic compounds, thereby exposing said sensor array to said volatile organic compounds.

17. (canceled)

18. (canceled)

19. (canceled)

20. (canceled)

21. (canceled)

22. The apparatus of claim 1, further comprising a heater, wherein said patch is disposed between said heater and said sensor array and wherein heating of said patch by said heater causes said sensor array to be exposed to volatile organic compounds that have been captured by said patch thereby causing interaction between said volatile organic compounds and said sensors.

23. (canceled)

24. The apparatus of claim 1, further comprising a smartphone having a camera that is disposed such that said colorimetric sensors are in said camera's field-of-view, wherein said smartphone is configured to output a normalized differential intensity for each primary color.

25. The apparatus of claim 1, further comprising a support vector machine that receives information indicative of which of said sensors have undergone interaction in response to exposure to a sample of volatile organic compounds, said support vector machine having been trained to recognize a condition associated with said information.

26. (canceled)

27. The apparatus of claim 1, further comprising an inner chamber, an outer chamber, and a heater, wherein said outer chamber encloses said heater, wherein said inner chamber is disposed on said heater and encloses said patch, which is heated by said heater to release volatile organic compounds and that also encloses said sensor array, which is exposed to said volatile organic compounds in said inner chamber, wherein said outer chamber comprises an aperture and a ring light disposed on said aperture to permit a camera to obtain an image of said sensor through said aperture.

28. A method comprising providing a machine-learning system that has been trained to use a profile of volatile organic compounds as a basis for determining the existence of a target condition, collecting volatile organic compounds from a subject onto a patch, providing a sensor array that comprises colorimetric sensors and chemo-resistive sensors disposed on fibrous substrate, exposing said sensor array to said volatile organic compounds, determining a volatilome of said subject based on a color change in said colorimetric sensors and a resistance change in said chemiresistive sensors.

29. (canceled)

30. (canceled)
